SEQUENCE LISTING (11 sequences)

SEQ ID NO: 1 - DNA, 972 bp, human
cgccgcgatggggctcctggactcggagccgggtagtgtcctaaacgtagtgtccacggcactcaacgacacggtagagttctaccgctggacctggtccatcgcagataagcgtgtggaaaattggcctctgatgcagtctccttggcctacactaagtataagcactctttatctcctgtttgtgtggctgggtccaaaatggatgaaggaccgagaaccttttcagatgcgtctagtgctcattatctataattttgggatggttttgcttaacctctttatcttcagagagttattcatgggatcatataatgcgggatatagctatatttgccagagtgtggattattctaataatgttcatgaagtcaggatagctgctgctctgtggtggtactttgtatctaaaggagttgagtatttggacacagtgttttttattctgagaaagaaaaacaaccaagtttctttccttcatgtgtatcatcactgtacgatgtttaccttgtggtggattggaattaagtgggttgcaggaggacaagcattttttggagcccagttgaattcctttatccatgtgattatgtactcatactatgggttaactgcatttggcccatggattcagaaatatctttggtggaaacgatacctgactatgttgcaactgattcaattccatgtgaccattgggcacacggcactgtctctttacactgactgccccttccccaaatggatgcactgggctctaattgcctatgcaatcagcttcatatttctctttcttaacttctacattcggacatacaaagagcctaagaaaccaaaagctggaaaaacagccatgaatggtatttcagcaaatggtgtgagcaaatcagaaaaacaactcatgatagaaaatggaaaaaagcagaaaaatggaaaagcaaaaggagattaaattgaactgggccttaactg

SEQ ID NO: 2 - protein, 314 aa, human
MGLLDSEPGSVLNVVSTALNDTVEFYRWTWSIADKRVENWPLMQSPWPTLSISTLYLLFVWLGPKWMKDREPFQMRLVLIIYNFGMVLLNLFIFRELFMGSYNAGYSYICQSVDYSNNVHEVRIAAALWWYFVSKGVEYLDTVFFILRKKNNQVSFLHVYHHCTMFTLWWIGIKWVAGGQAFFGAQLNSFIHVIMYSYYGLTAFGPWIQKYLWWKRYLTMLQLIQFHVTIGHTALSLYTDCPFPKWMHWALIAYAISFIFLFLNFYIRTYKEPKKPKAGKTAMNGISANGVSKSEKQLMIENGKKQKNGKAKGD

SEQ ID NO: 3 - DNA, 967 bp, human
cgccgcgatggggctcctggactcggagccgggtagtgtcctaaacgtagtgtccacggcactcaacgacacggtagagttctaccgctggacctggtccatcgcagataagcgtgtggaaaattggcctctgatgcagtctccttggcctacactaagtataagcactctttatctcctgtttgtgtggctgggtccaaaatggatgaaggaccgagaaccttttcagatgcgtctagtgctcattatctataattttgggatggttttgcttaacctctttatcttcagagagttattcatgggatcatataatgcgggatatagctatatttgccagagtgtggattattctaataatgttcatgaagtcaggatagctgctgctctgtggtggtactttgtatctaaaggagttgagtatttggacacagtgttttttattctgagaaagaaaaacaaccaagtttctttccttcatgtgtatcatcactgtacgatgtttaccttgtggtggattggaattaagtgggttgcaggaggacaagcattttttggagcccagttgaattcctttatccatgtgattatgtactcatactatgggttaactgcatttggcccatggattcagaaatatctttggtggaaacgatacctgactatgttgcaactgattcaattccatgtgaccattgggcacacggcactgtctctttacactgactgccccttccccaaatggatgcactgggctctaattgcctatgcaatcagcttcatatttctctttcttctacattcggacatacaaagagcctaagaaaccaaaagctggaaaaacagccatgaatggtatttcagcaaatggtgtgagcaaatcagaaaaacaactcatgatagaaaatggaaaaaagcagaaaaatggaaaagcaaaaggagattaaattgaactgggccttaactg

SEQ ID NO: 4 - protein, 271 aa, human
MGLLDSEPGSVLNVVSTALNDTVEFYRWTWSIADKRVENWPLMQSPWPTLSISTLYLLFVWLGPKWMKDREPFQMRLVLIIYNFGMVLLNLFIFRELFMGSYNAGYSYICQSVDYSNNVHEVRIAAALWWYFVSKGVEYLDTVFFILRKKNNQVSFLHVYHHCTMFTLWWIGIKWVAGGQAFFGAQLNSFIHVIMYSYYGLTAFGPWIQKYLWWKRYLTMLQLIQFHVTIGHTALSLYTDCPFPKWMHWALIAYAISFIFLFLLHSDIQRA

SEQ ID NO: 5 - DNA, 970 bp, human
cgccgcgatggggctcctggactcggagccgggtagtgtcctaaacgtagtgtccacggcactcaacgacacggtagagttctaccgctggacctggtccatcgcagataagcgtgtggaaaattggcctctgatgcagtctccttggcctacactaagtataagcactctttatctcctgtttgtgtggctgggtccaaaatggatgaaggaccgagaaccttttcagatgcgtctagtgctcattatctataattttgggatggttttgcttaacctctttatcttcagagagttattcatgggatcatataatgcgggatatagctatatttgccagagtgtggattattctaataatgttcatgaagtcaggatagctgctgctctgtggtggtactttgtatctaaaggagttgagtatttggacacagtgttttttattctgagaaagaaaaacaaccaagtttctttccttcatgtgtatcatcactgtacgatgtttaccttgtggtggattggaattaagtgggttgcaggaggacaagcattttttggagcccagttgaattcctttatccatgtgattatgtactcatactatgggttaactgcatttggcccatggattcagaaatatctttggtggaaacgatacctgactatgttgcaactgattcaattccatgtgaccattgggcacacggcactgtctctttacactgactgccccttccccaaatggatgcactgggctctaattgcctatgcaatcagcttcatatttctctttctaactctacattcggacatacaaagagcctaagaaaccaaaagctggaaaaacagccatgaatggtatttcagcaaatggtgtgagcaaatcagaaaaacaactcatgatagaaaatggaaaaaagcagaaaaatggaaaagcaaaaggagattaaattgaactgggccttaactg

SEQ ID NO: 6 - protein, 272 aa, human
MGLLDSEPGSVLNVVSTALNDTVEFYRWTWSIADKRVENWPLMQSPWPTLSISTLYLLFVWLGPKWMKDREPFQMRLVLIIYNFGMVLLNLFIFRELFMGSYNAGYSYICQSVDYSNNVHEVRIAAALWWYFVSKGVEYLDTVFFILRKKNNQVSFLHVYHHCTMFTLWWIGIKWVAGGQAFFGAQLNSFIHVIMYSYYGLTAFGPWIQKYLWWKRYLTMLQLIQFHVTIGHTALSLYTDCPFPKWMHWALIAYAISFIFLFLTLHSDIQRA

SEQ ID NO: 7 - DNA, 1292 bp, mouse
cagtcgcccacggtccatcggagcctctcttctcgcccgcttgtcgtacctctcctcgccaagatggggctgctggactcagagcccggcagcgtcctgaacgcgatgtccacggcattcaacgacaccgtggagttctatcgctggacctggaccattgcagataaacgtgtagcagactggccgctgatgcagtctccatggccaacgataagcataagcacgctctatctcctgttcgtgtggctgggtccaaagtggatgaaagaccgcgagccgttccaaatgcgcttagtactcataatctataattttggcatggttttgcttaaccttttcatcttcagagagctattcatgggatcatacaacgcaggatacagctatatttgccagtcagtggattattctaatgatgttaatgaagtcaggatagcggcggccctgtggtggtattttgtatcgaaaggcgttgagtatttggacacagtgttttttatcctgaggaagaaaaacaaccaagtctccttccttcacgtgtaccaccactgcaccatgttcactctgtggtggattggaatcaagtgggtggctggaggccaagcgtttttcggggcccagatgaactctttcatccacgtgatcatgtactcctactatgggctgactgcgttcggcccctggatccagaaatatctttggtggaagcgatacctgaccatgctgcagctggtccagttccacgtgaccatcggacacacagcactgtctctctacaccgactgccccttccccaagtggatgcactgggctctgatcgcctacgccatcagcttcatcttcctcttcctcaacttctacactcggacatacaatgagccgaagcagtcaaaaaccggaaagacggccacgaatggtatctcatcgaacggcgtgaataaatcagagaaagcgttagaaaacgggaaaccccagaaaaacgggaagccaaaaggagagtaaattgaactgggccttaaccggtagacagtgaggaaactcctgtgtcattttaaaaagttcaggggcaacagaagcagagggtctgggctggggagaaaggcagatagggtctttgcccttcagactgagtaaaacttttcaatatatggtacccagatgttttatttatgaagtttttattttaaaagttttttttttattaacccttcatgttgtccaaaaccaaagcaacccccaatgtggaccttgggagccttttctctgttaacattccgccttgggcaatgggg

SEQ ID NO: 8 - protein, 312 aa, mouse
MGLLDSEPGSVLNAMSTAFNDTVEFYRWTWTIADKRVADWPLMQSPWPTISISTLYLLFVWLGPKWMKDREPFQMRLVLIIYNFGMVLLNLFIFRELFMGSYNAGYSYICQSVDYSNDVNEVRIAAALWWYFVSKGVEYLDTVFFILRKKNNQVSFLHVYHHCTMFTLWWIGIKWVAGGQAFFGAQMNSFIHVIMYSYYGLTAFGPWIQKYLWWKRYLTMLQLVQFHVTIGHTALSLYTDCPFPKWMHWALIAYAISFIFLFLNFYTRTYNEPKQSKTGKTATNGISSNGVNKSEKALENGKPQKNGKPKGE

SEQ ID NO: 9 - DNA, 25 bp, mouse
ctccgcagataaacgtgtagcagac

SEQ ID NO: 10 - DNA, 25 bp, mouse
agagtgccgttaacaaacctacctc

SEQ ID NO: 11 - DNA, 27 bp, Artificial Sequence
  A sequence designed to detect the deletion of the mouse gene
gggtgggattagataaatgcctgctct

(19) United States
(12) Patent Application Publication
(10) Pub. No.: US 2005/0262580 A1
(43) Pub. Date: Nov. 24, 2005

(54) Transgenic mouse with a targeted deletion of Elovl4 gene

(76) Inventors: Konstantin Petrukhin, Phoenixville, PA (US); Andrea L. Webber, Yardley, PA (US)

Correspondence Address: MERCK AND CO., INC, P O BOX 2000, RAHWAY, NJ, 07065-0907, US

(21) Appl. No.: 11/058,817

(22) Filed: Feb. 16, 2005

Related U.S. Application Data
(60) Provisional application No. 60/545,358, filed on Feb. 18, 2004.

(51) Int. Cl. A01K 67/027; C12N 5/06

(52) U.S. Cl. 800/18; 435/354

(57) ABSTRACT

The present invention relates to a transgenic mouse whose genome is heterozygous for a disruption of a native Elovl4 gene. The transgenic mouse and cells derived therefrom can be used to screen drug or food supplement for the treatment and prevention of visual disorders such as Stargardt-like dominant macular dystrophy.

### CROSS-REFERENCE TO RELATED APPLICATIONS

This application claims benefit to U.S. provisional application No. 60/545,358 filed on Feb. 18, 2004.

### STATEMENT REGARDING FEDERALLY-SPONSORED R&D

none

### REFERENCE TO MICROFICHE APPENDIX

none

### FIELD OF THE INVENTION

The present invention relates to a transgenic mouse with a targeted deletion of Elovl4 gene, and the uses of the transgenic mouse in the screening of drug candidates and food supplements.

### BACKGROUND OF THE INVENTION

Macular dystrophy is a term applied to a heterogeneous group of diseases that collectively are the cause of severe visual loss in a large number of people. A common characteristic of macular dystrophy is a progressive loss of central vision resulting from the degeneration of photoreceptor cells in the retinal macula. In many forms of macular dystrophy, the end stage of the disease results in legal blindness. More than 20 types of macular dystrophy are known: e.g., age-related macular dystrophy, Stargardt-like dominant macular dystrophy (STGD3, MIM 600110), recessive Stargardt's disease, atypical vitelliform macular dystrophy (VMD1), Usher Syndrome Type 1B, autosomal dominant neovascular inflammatory vitreoretinopathy, familial exudative vitreoretinopathy, and Best's macular dystrophy (also known as hereditary macular dystrophy or Best's vitelliform macular dystrophy (VMD2). For a review of the macular dystrophies, see Sullivan & Daiger, Mol. Med. Today 2:380-386 (1996).

Stargardt-like dominant macular dystrophy, also called autosomal dominant macular atrophy (adMD), is a juvenile-onset macular degeneration. Patients afflicted with this disease generally have normal vision as young children, but during childhood, visual loss begins, which rapidly progresses to legal blindness. Clinically it is characterized by the presence of an atrophic macular lesion with sharp borders and is often associated with yellow fundus flecks. The gene responsible for Stargardt-like macular dystrophy has been identified, along with its normal allelic form, ELOVL4 (for elongation of very long chain fatty acids). The mutant gene encodes a mutant protein containing a frame-shift mutation, resulting in abnormal fatty acid synthesis and transport in the retina (Zhang, et al. Nature Genetics 27:89-93 (2001); WO0187921). The exact functions of ELOVL4 are unknown yet.

Moreover, the pathological features seen in Stargardt-like dominant macular dystrophy are in many ways similar to the features seen in age-related macular dystrophy (AMD), which is the leading cause of blindness in older patients in the developed world. A significant proportion of the AMD cases is caused by recessive mutations in the recessive Stargardt disease gene. (Allikmets, et al, Science 277:1805-1807 (1997)).

The genetic study of AMD, however, is extraordinarily difficult, because AMD patients are diagnosed when they become symptomatic in old age. When the patients are diagnosed, their parents are usually no longer living and their children are still asymptomatic. Thus, family studies, which are used to study the genetic basis of many inherited diseases, are not practical for age-related macular dystrophy. As there are currently no widely effective treatments for atrophic AMD, it is hoped that study of Stargardt-like dominant macular dystrophy, and in particular the discovery of the underlying genetic cause of Stargardt-like dominant macular dystrophy, will shed light on age-related macular dystrophy as well.

There is a need to study the functions of ELOVL4, to develop the treatment of Stargardt-like dominant macular dystrophy, and other visual diseases.

The references cited herein are not admitted to be prior art to the claimed invention.

### SUMMARY OF THE INVENTION

The present invention provides a transgenic mouse whose genome is heterozygous for a disruption of a native Elovl4 gene. The transgenic mouse produces an average triglyceride level at about 1.8 fold of that of a wild type mouse homozygous for the native Elovl4 gene. According to a preferred embodiment of the present invention, the transgenic mouse is fertile.

The present invention provides a cell line established from the transgenic mouse.

The present invention provides a method for producing a mouse whose genome is heterozygous for a disruption of a native Elovl4 gene. The method comprises: a) providing a DNA sequence which targets and inserts a disruption into a Elovl4 gene; b) introducing the DNA sequence into mouse ES cells; c) selecting those mouse ES cells whose genome comprise a disruption of a Elovl4 gene; d) introducing an ES cell selected in step c) in a mouse blastocyst; e) transplanting the blastocyst of step d) into a foster mother mouse; f) developing the transferred blastocyst to term to produce chimeric mice; and g) mating the chimeric mice to produce a mouse heterozygous for a disruption of the Elovl4 gene. The heterozygous mouse produces an average triglyceride level at about 1.8 fold of that of a wild type mouse homozygous for a native Elovl4 gene.

According to an embodiment of the present invention, the genome of the transgenic mouse further comprises a transgene comprising a DNA sequence encoding a non-native Elovl4 operably linked to a promoter selected from the group consisting of neural and neuronal specific promoters, wherein the mouse is viable. The promoter can be selected from the group consisting of mouse Elovl4 promoter, mouse rod opsin promoter, and mouse IRBP promoter. The non-native Elovl4 can be a human ELOVL4, or a mutant of human ELOVL4. According to a preferred embodiment of the present invention, the mutant of human ELOVL4 encodes SEQ ID NO:4 or SEQ ID NO:6.

The present invention provides an assay for determining the effect of a compound on Elov4. The method comprises providing a male and female heterozygous Elovl4 knockout mouse; exposing the female mouse to a compound; breeding the male and female mouse to produce offspring; and determining the genotypes of the offspring. According to an embodiment of the present invention, the exposing is administering. According to a preferred embodiment of the present invention, the compound is administered to the female mouse intramuscularly, intravenously, subcutaneously, orally, rectally, or percutaneously. According to an alternative embodiment of the present invention, the exposing is feeding. According to a preferred embodiment of the present invention, the compound is DHA or ARA.

The present invention also provides an assay for screening a compound capable of lowering triglyceride level. The method comprises, exposing a heterozygous Elovl4 knockout mouse to a compound, and determining the triglyceride level of the mouse. According to a preferred embodiment of the present invention, the compound is selected from the group consisting of niacin, pantethine, fibrates (PPARalpha agonists), and statins.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1 is normal human ELOVL4 cDNA sequence (SEQ ID NO: 1) and the amino acid sequence (SEQ ID NO: 2) of the human ELOVL4 protein. Underlined nucleotides in bold encompassing base pairs 797-801 represent the deletion found in patients with dominant Stargardt-like macular dystrophy. The protein fragment deleted in patients with Stargardt-like macular dystrophy is shown in bold underline.

FIG. 2A shows the structures of docosahexaenoic acid (DHA), arachidonic acid (ARA), Linoleic acid, and a-Linolenic acid (ALA). DHA is indicated as 22:6 (n-3), where the first number indicates chain length, the second number indicates the number of double bonds, and “n-3” indicates the position of the first double bond as its relates to the terminal methyl group.

FIG. 2B depicts the enzymatic conversions involved in the linoleic acid (n-3) and a-linolenic acid (n-6) pathways of essential fatty acid synthesis, including three elongation steps required of the biosynthesis of DHA.

FIG. 3 is the cDNA sequence (SEQ ID NO: 3) and the amino acid sequence (SEQ ID NO: 4) of the human ELOVL4 797-801ΔAACTT mutant. The region of the protein encompassing amino acids 264-271 (bold underlined) represent a fragment generated as a result of the 5-base pair deletion in patients with dominant Stargardt-like macular dystrophy.

FIG. 4 is the cDNA sequence (SEQ ID NO: 5) and the amino acid sequence (SEQ ID NO: 6) of the human ELOVL4 796ΔT/800ΔT mutant. The region of the protein encompassing amino acids 264-272 (bold underlined) represent a fragment generated as a result of the double 1-base pair deletions in patients with dominant Stargardt-like macular dystrophy.

FIG. 5 is murine Elovl4 cDNA sequence for (SEQ ID NO: 7) and the amino acid sequence (SEQ ID NO: 8) of the murine Elovl4 protein. The region of the cDNA sequence encompassing base pairs 195-256 (bold underlined) represent the 62 bp deletion of Elovl4 exon 2 in the targeting vector used to disrupt Elovl4 gene.

FIG. 6 shows the pairwise comparison of human and mouse Elvol4 proteins. The upper amino acid sequence shown is the human ELOVL4 protein (SEQ.ID.NO. 2). The lower amino acid sequence shown is the mouse Elovl4 protein (SEQ.ID.NO. 8). The two proteins are highly identical which indicates they are true orthologues. Both proteins share the cytosolic carboxy-terminal dilysine motif responsible for the retrieval of transmembrane proteins from cis-Golgi to the endoplasmic reticulum (two lysines are located at −3 and −5 positions with respect to the carboxyl terminus).

FIG. 7 is a schematic representation of a genomic map of a portion of the murine Elovl4 gene including exon 2 and the disruption of the mouse chromosomal Elovl4 gene by targeted recombination using targeting vector. The striped bars are mouse genomic sequence beyond targeting cDNA sequence. The pA is the polyadenylation signal that should truncate transcripts at the position. Numbers refer to base pair positions in the targeting cDNA sequence.

### DETAILED DESCRIPTION OF THE INVENTION

### 1. ELOVL4 and Its Orthologs

1.1. The human ELOVL4

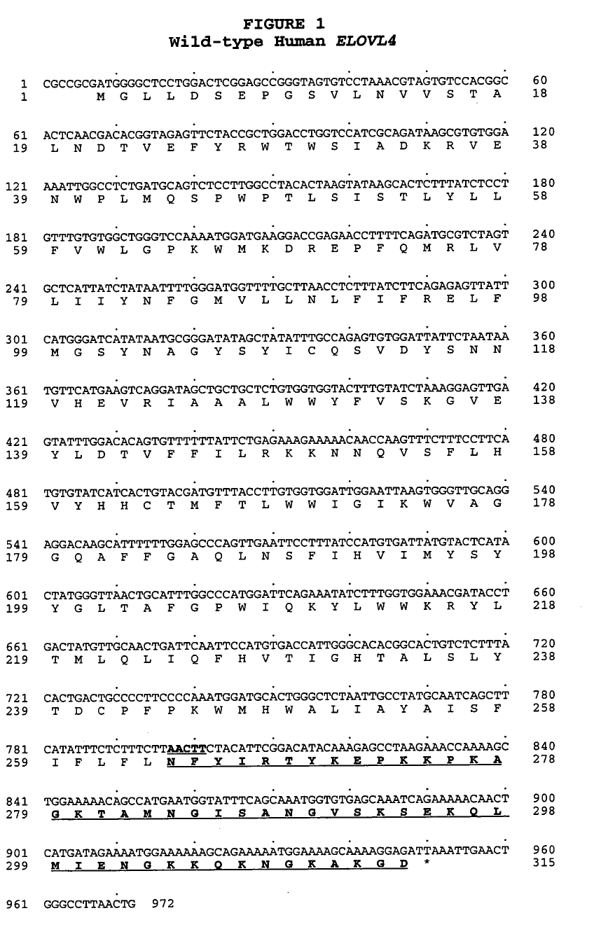

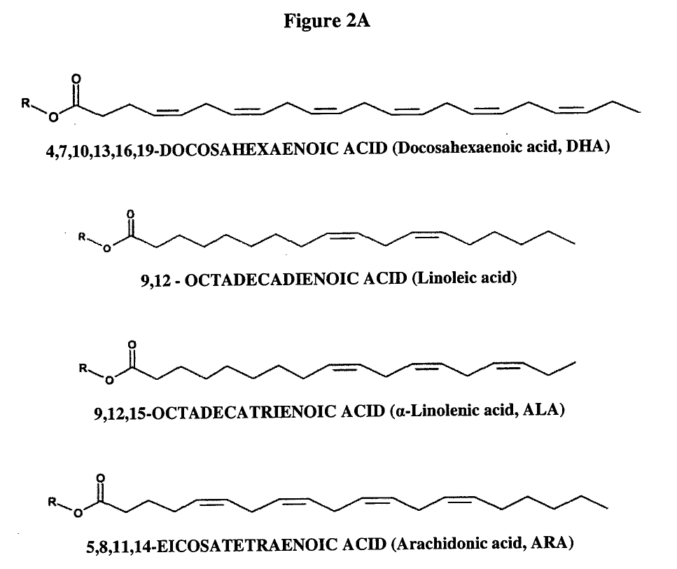

The human ELOVL4 gene (SEQ ID NO: 1) encodes a putative protein of 314 amino acids (SEQ ID NO:2) (FIG. 1) with approximately 35% amino acid identity to members of the ELO gene family, which encode components of the membrane-bound fatty acid elongation system. ELO proteins have been identified in yeast (Oh, C. S. et al. J. Biol. Chem. 272:17376-17384 (1997)), and rodent (Tvrdik, P. et al. J. Biol. Chem. 272:31738-31746 (1997); Tvrdik, P. et al. J. Cell Biol. 149:707-318 (2000)).

Similar to other members of the ELO family, Human ELOVL4 has three features: a hydropathy plot that predicts five transmembrane segments; a single HXXHH motif identified with fatty acid desaturase and other dioxy iron cluster proteins; and a strong signal (dilysine motif with the lysines at positions −3 and −5 relative to the carboxy terminus) shown to be responsible for the retention of transmembrane proteins in the endoplasmic reticulum, the known site of biosynthesis of fatty acids with very long chains (Zhang, K. et al. Nature Genetics 27:89-93 (2001)). ELOVL4 has a specific and abundant expression in human retina, as well as lower expressions in brain and testis. In the adult retina, ELOVL4 is expressed exclusively in photoreceptor cells, both rod and cone photoreceptors. (ibid.).

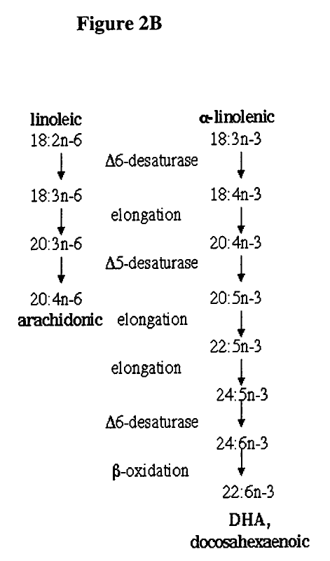

ELOVL4 may play a key role in the synthesis of certain essential fatty acids. Essential fatty acids are polyunsaturated fatty acids that cannot be de novo synthesized by mammals, yet are required for a number of important biochemical processes. Thus, essential fatty acids must be supplied either directly in the diet, or synthesized from dietary essential fatty acids, such as linoleic acid and alpha-linolenic acid (ALA). These two dietary EFAs undergo a number of biosynthetic reactions that convert them into various other EFAs. The reactions include a series of alternating reactions involving the removal of two hydrogens coupled with the insertion of an additional double bond (desaturation) and the lengthening of the fatty acid chain by the addition of two carbons (chain elongation). Through the pathways, docosahexaenoic acid (DHA), an essential fatty acid of omega-3 family, is synthesized from alpha-linolenic acid (ALA), while arachidonic acid (ARA) is synthesized from linoleic acid (FIGS. 2A and 2B). It is believed that ELOVL4 is involved in one of three elongation steps required for DHA synthesis (FIG. 2B).

1.2. Docosahexaenoic Acid (DHA)

DHA is a highly polyunsaturated, long-chain fatty acid, which has six double bonds and is 22 carbons in length (FIG. 2A). DHA is a critical component of membranes in vertebrate retina and in neural tissues, especially in the grey matter of the brain, comprising 30-50% of all fatty acids in these tissues (Salem, N. J. and Yergey, J. A. In Health Effects of Polyunsaturated Fatty Acids in Seafood, pp. 263-317, Academic Press, New York (1986)).

DHA is an important structural component of cellular membrane where it enhances membrane fluidity for high density packing of proteins, and lateral compressibility (Treen, M. et al. Archives of Biochemistry and Biophysics 294:564-570 (1992)); Koening B. W. et al. Biophysics Journal 73:1954-1966 (1997)). DHA is also involved in various signal transduction pathway and plays important roles in both induction and inhibition of apoptosis (See, e.g., Rotstein, N. P. et al. Investigative Ophthalmology and Visual Science 44:2252-2259 (2003); Diep, Q. N. et al. Hypertension 36:851-855 (2000)).

DHA is required for normal vision development. Loss of brain DHA results in the loss of many sensory, behavioral, and cognitive functions both in animals and human. Using a dog model, it was demonstrated that DHA deficiency causes retinal degeneration (Alvarez R A et al. Invest Ophthalmol Vis Sci. 35(2):402-8 (1994)). DHA also appears to be important for the development of central nervous system. Studies suggested that brain DHA deficiency reduces neuron size in the hippocampus, hypothalamus, piriform cortex, and parietal cortex (Ahmad, A. et al. Pediatric Neurology 26:210-218 (2002)).

WTO recommends the supplementation of infant formulas with human milk levels of DHA and ARA. The need for preformed DHA and ARA may be particularly important in preterm infants, because they may have suboptimal capability to elongate and desaturate dietary essential fatty acids to their respective long-chain derivatives, such as ARA and DHA (Burns, R. A., et al. Food and Chemical Toxicology 37:23-36 (1999)).

DHA can also decrease the risk for coronary heart disease. Increased plasma triglyceride level is a risk factor for coronary heart disease. As discussed above, DHA is a highly polyunsaturated, very long-chain fatty acid (VLCFA). DHA represents the most abundant component of fish oil. Dietary DHA/fish oil is thought to suppress endogenous VLCFA synthesis. Suppression of the n-6 biosynthetic pathway may result in reduced production of arachidonic acid (ARA). Reduced production of ARA is thought to be a mediator of the very well documented beneficial effect of fish oil/DHA in preventing hypertriglyceridemia and cardiovascular disease.

1.3. The ELOVL4 Mutants and Orthologs

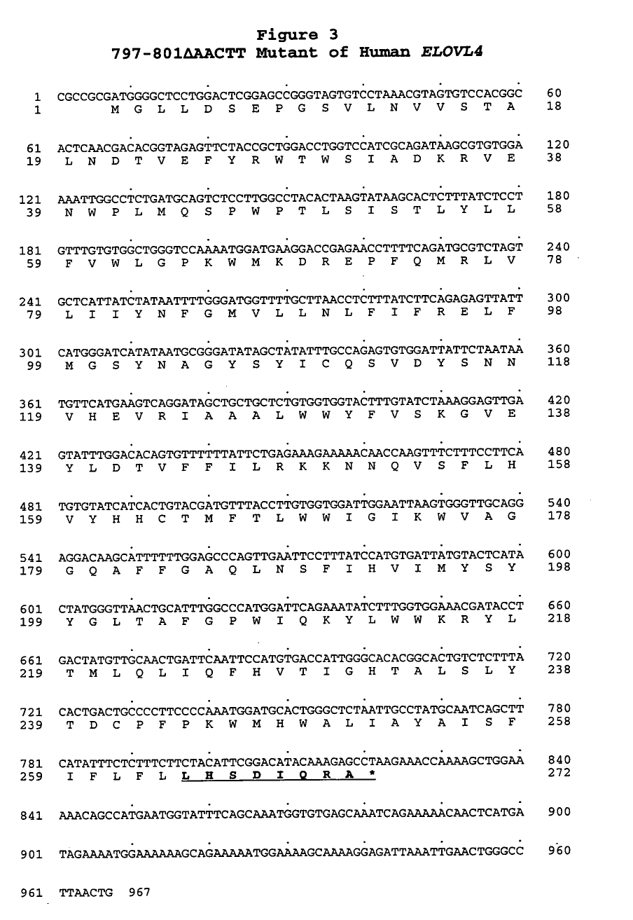

Analysis of four STGD3 kindreds and one family with adMD revealed an ELOVL4 mutant. The ELOVL4 mutant (SEQ ID NO:3) has a 5-bp deletion in exon 6 (FIG. 3) starting at position 797 of the ELOVL4 cDNA. As used herein, the mutant is designated 797-801ΔAACTT. The deletion results in a frameshift, loss of a fragment of 51 amino acids at the C terminus including a dilysine targeting signal, and synthesis of an aberrant peptide from amino acid 264 to 271, followed by a premature stop codon (SEQ ID NO:4) (FIG. 3). This would prevent ELF protein from trafficking to the site of biosynthesis of very long chain fatty acids (membranes of the endoplasmic reticulum) (ibid.). An independent study of STGD3 kindreds demonstrated that the patients carry the same ELOVL4 mutant, 797-801ΔAACTT (Vrabec, T. R., et al. American Journal of Ophthalmology, 136:542-545 (2003)).

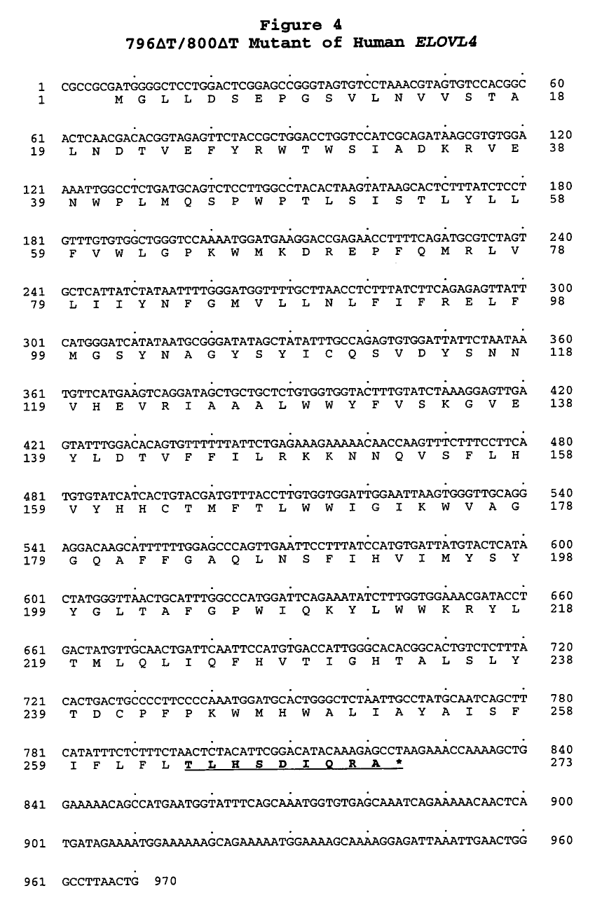

Furthermore, study of STGD3 patients also identified another ELOVL4 mutant, which contains two 1-bp deletions separated by four nucleotides in exon 6 of the ELOVL4 gene, the same location of the 797-801ΔAACTT mutation (SEQ ID NO:5). The mutation results in a frameshift and the truncation of the ELOVL4 protein (SEQ ID NO:6) (FIG. 4), similar to the effect of the 5-bp deletion, 797-801ΔAACTT. (Bernstein, P. S., et al. Investigative Ophthalmology & Visual Science 42:3331-3336 (2001)). As used herein, the mutant of double 1-bp deletions is designated 796ΔT/800ΔT.

Orthologs of human ELOVL4 have been identified from mammalian to invertebrate species. Orthologs are genes (or proteins) in different species that evolved from a common ancestral gene (or protein). Typically, orthologs have the same functions in the different species. The orthologs and human ELOVL4 are highly conserved in sequence and expression pattern, suggesting functional conservation of Elovl4 during evolution (Zhang, K. et al. Nature Genetics 27:89-93 (2001); Zhang X-M, et al. Molecular Vision 9:301-307 (2003); Lagali P. S., et al. Investigative Ophthalmology & Visual Science 44:2841-2850 (2003)).

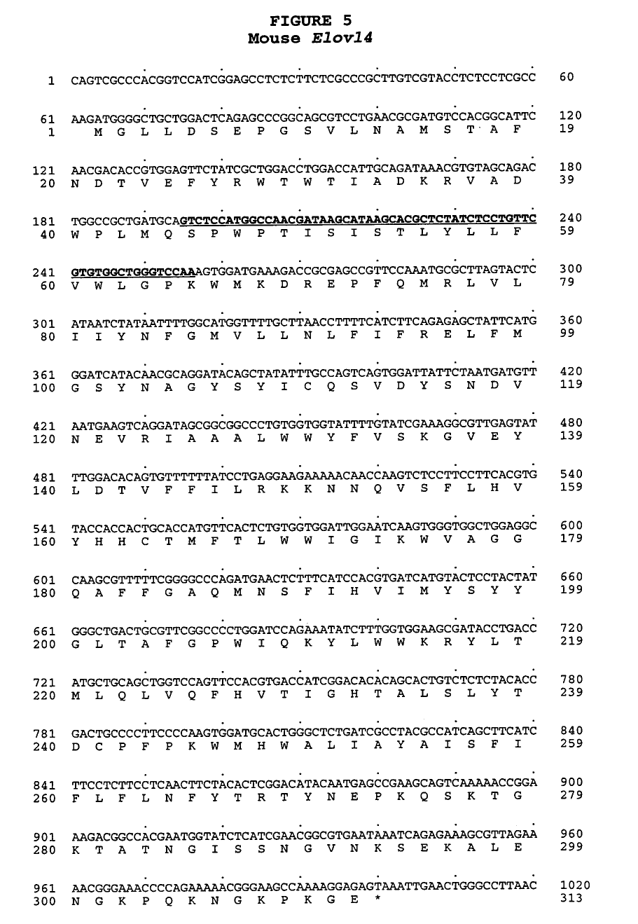

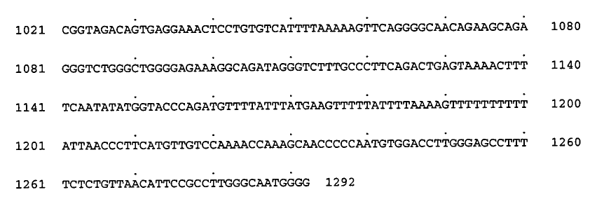

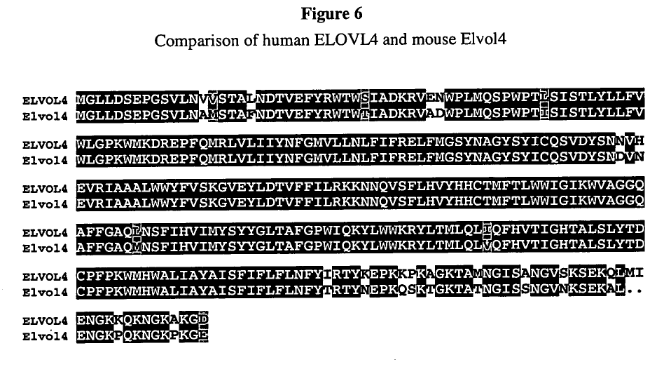

Among the ELOVL4 orthologs, the mouse Elovl4 (SEQ ID NO:8) (FIG. 5) shares strong homology of amino acid sequence with human ELOVL4, i.e., 92% amino acid identity (FIG. 6) (Zhang, K. et al. Nature Genetics 27:89-93 (2001)). In the developing retina of mouse, Elovl4 expression switches from predominant ganglion cell expression in embryonic and early postnatal development to predominant expression in the photoreceptor inner segments in later stages (Zhang X-M, et al. Molecular Vision 9:301-307 (2003)).

### 2. The Transgenic Mouse with a Targeted Disruption of Elovl4

The present invention relates to a transgenic animal carrying an altered Elovl4 gene. The transgenic animal may lack one or both native Elovl4 alleles (Elovl4 null), and may express a non-native Elovl4 protein.

As used herein, “animal” refers to all non-human mammals in all stages of development, including embryonic and fetal stages. A “transgenic animal” is an animal containing one or more cells bearing genetic information introduced by deliberate genetic manipulation at a subcellular level, such as by microinjection or infection with recombinant virus. This introduced genetic information can be DNA fragment integrated within a chromosome, or it can be extra-chromosomally replicating DNA molecule. Unless otherwise noted or understood from the context of the description of an animal, the term “transgenic animal” as used herein refers to a transgenic animal in which the genetic information was introduced into a germ line cell, thereby conferring the ability to transfer the information to offspring. If offspring in fact possess some or all of the genetic information, then they, too, are transgenic animals. The genetic information is typically provided in the form of a transgene carried by the transgenic animal.

The genetic information received by the animal can be foreign to the species of animal to which the recipient belongs, or foreign only to the particular individual recipient. In the latter case, the information can be altered or it can be expressed differently than the native gene. Alternatively, the altered or introduced gene can cause the native gene to become non-functional to produce a “knockout” animal.

As used herein, a “targeted gene” or “Knockout” (KO) is a DNA sequence introduced into the germline of a non-human animal by genetic manipulation, such as the methods described herein. The targeted genes of the invention include nucleic acid sequences which are designed to specifically alter cognate endogenous alleles.

The transgenic animals of the present invention are generated utilizing a partial or full-length Elovl4 coding sequence. An Elovl4 gene that naturally occurs in the animal is referred to as the native gene, and if it is not mutant, it can also be referred to as wild-type. The alterations to a native gene include modifications, deletions and substitutions, which can render the native gene nonfuctional, producing a “knockout” animal, or can lead to an Elovl4 with altered expression or activity.

An altered Elovl4 gene should not fully encode the same Elovl4 as native to the host animal, and its expression product can be altered to a minor or great degree, or absent altogether. In cases where it is useful to express a non-native Elovl4 gene in a transgenic animal in the absence of a native Elovl4 gene, we prefer that the altered Elovl4 gene induce a null lethal knockout phenotype in the animal. However a more modestly modified Elovl4 gene can also be useful and is within the scope of the present invention.

A type of target cell for transgene introduction is the embryonic stem cell (ES). ES cells can be obtained from pre-implantation embryos cultured in vitro and fused with embryos (M. J. Evans et al, Nature 292:154-156 (1981); Bradley et al., Nature 309:255-258 (1984); Gossler et al. Proc. Natl. Acad. Sci. USA 83:9065-9069 (1986); and Robertson et al., Nature 322:445-448 (1986)). Transgenes can be efficiently introduced into the ES cells by a variety of standard techniques such as DNA transfection, microinjection, or by retrovirus-mediated transduction. The resultant transformed ES cells can thereafter be combined with blastocysts from a non-human animal. The introduced ES cells thereafter colonize the embryo and contribute to the germ line of the resulting chimeric animal (R. Jaenisch, Science 240: 1468-1474 (1988)).

The functions of Elovl4 can be examined in a variety of ways. One approach to the problem of determining the contributions of individual genes and their expression products is to use isolated genes to selectively inactivate the native wild-type gene in totipotent ES cells (such as those described herein) and then generate transgenic mice. Techniques are available to inactivate or alter any genetic region to any mutation desired by using targeted homologous recombination to insert specific changes into chromosomal alleles. The use of gene-targeted ES cells in the generation of gene-targeted transgenic mice was described in 1987 (Thomas et al., Cell 51:503-512, (1987)) and is reviewed elsewhere (Frohman et al., Cell 56:145-147. (1989); Capeccbi, Trends in Genet. 5:70-76 (1989); Baribault et al., Mol. Biol. Med. 6:481-492, (1989); Wagner, EMBO J. 9:3025-3032 (1990); Bradley et al., BioTechnology 10:534-539 (1992)).

The methods for evaluating the targeted recombination events as well as the resulting knockout mice are readily available and known in the art. Such methods include, but are not limited to DNA (Southern) hybridization to detect the targeted allele, polymerase chain reaction (PCR), polyacrylamide gel electrophoresis (PAGE), and Western blots to detect DNA, RNA and protein.

The murine Elovl4 gene is at least 27.5 kb in size and is encoded by six exons. Mouse genome fragment from chromosome 9 containing Elovl4 is represented in GenBank accession number NT_{—}039475; coordinates of the Elovl4 gene within this fragment are: 3413036-3440445. Coordinates of the six exons of murine Elovl4 are as follows: 3413036-3415152 for exon 1, 3417479-3417606 for exon 2, 3419378-3419549 for exon 3, 3422612-3422692 for exon 4, 3424320-3424507 for exon 5, and 3440284-3440445 for exon 6. The murine Elovl4 cDNA is about 2.9 kb. An animal without an active Elovl4 gene could be used to evaluate the role of Elovl4 in vision development and in the central nervous system. However, it was not known if such an animal could be produced, e.g., if such an animal be viable.

Herein, heterozygous mouse for a disruption of a native Elovl4 gene is viable. The heterozygous mouse has an average triglyceride level at about 1.8 fold of that of the wild type mouse.

In contrast, homozygous Elovl4 knock-out mice cannot be produced through breeding of the heterozygous mice, indicating a lethal phenotype of Elovl4 null-mutant. The results suggest that Elovl4 play key roles not only in the vision development, but also in the development of other systems, most likely in the central nervous system. Mutations in genes involved in long chain fatty acid metabolism have been implicated in several central nervous system diseases (see, e.g., Mosser, J. et al. Nature 361:726-730 (1993); Braun, A. et al. Am J Hum Genet 56:854-861 (1995); Kaul R., et al. Nat Genet 5:118-123 (1993)). However, ELOVL4 is the first identified gene possibly involved in this biosynthetic pathway linked to retinal photoreceptor degeneration (Zhang, et al. Nature Genetics 27:89-93 (2001); Zhang X-M, et al. Molecular Vision 9:301-307 (2003)).

### 3. The Utilities of the Transgenic Mice

The transgenic animal of the invention can be used in the study of the expression and activity of the Elovl4 gene and protein, modulators of the activity of the Elovl4 gene or protein, and aspects of visual disorders, e.g., Stargardt-like dominant macular dystrophy, and disorders involving the central nervous system. The present invention provides a useful animal model by which to validate immunological reagents designed to detect the Elovl4 protein.

3.1. The Expression of Human ELOVL4 Allele in the Elovl4 Knockout Mice

The heterozygous Elovl4 knockout mice can facilitate production of allelic series of Elovl4. Non-native alleles of Elovl4 may be introduced into the heterozygous mice either at the genomic Elovl4 locus via gene-targeting, or at other genomic sites using transgenic pronuclear injection. For example, a human ELOVL4 allele can be introduced into the Elovl4 mutant background. The ELOVL4 allele can be genomic DNA or cDNA sequence. The ELOVL4 allele may encode wild-type or mutant ELOVL4 such as 797-801ΔAACTT or 796ΔT/800ΔT. The promoter for the non-native allele can be that for the murine Elovl4 or other appropriate promoters, including 221-bp fragment of the mouse rod opsin promoter (Quiambao, A. B. Vis Neurosci. 14(4):617-25 (1997)) or 5′ flanking region of the mouse interphotoreceptor retinoid-binding protein (IRBP) gene. (Borst, D. E. et al. Current Eye Research, 2001, Vol. 23, No. 1, pp. 20-32).

As discussed above, the mouse Elovl4 shares strong homology of amino acid sequence and similar expression pattern with human ELOVL4 mutant, suggesting functional conservation of Elovl4 during evolution. Hence, the transgenic mice carrying a murine Elovl4 allele and a human ELOVL4 mutant should develop a syndrome similar to Stargardt-like dominant macular dystrophy in human. Such an animal disease model can be used to study Stargardt-like dominant macular dystrophy, and screen drugs or food supplements for the prevention and treatment of the disease.

Moreover, the wild-type human ELOVL4 allele should be capable of rescuing the homozygous Elovl4 knockout mice from the lethality phenotype. Such a “humanized” mouse without endogenous mouse Elovl4 protein can be produced by the cross of heterozygous Elovl4 knockout mice that carry a wild-type human ELOVL4 allele with those that do not carry any non-native Elovl4 allele. The “humanized” mouse may be used to study evolutionary conservation of ELOVL4 functions and its structure-function relationship, and screen for pharmacological agents that modify function or phenotypic pathophysiology of ELOVL4. The “humanized” mouse is also useful for the establishment of a non-human model for diseases involving Elovl4, such as Stargardt-like dominant macular dystrophy.

The present invention provides an animal model useful in the design and assessment of various approaches to modulating ELOVL4 activity and expression. Such modified transgenic non-human animals can also be used as a source of cells for cell culture. These cells can be used for corresponding in vitro studies of ELOVL4 expression, activity and the modulation thereof.

3.2. The Screening of Compounds Capable of Rescuing Elovl4 Null-Mutant

As discussed above, Elovl4 is believed to play key roles in the biosynthesis of essential fatty acids, such as DHA. The lethality phenotype of homozygous Elovl4 knockout mice may also be rescued with the administration of certain compounds to the parental homozygous Elovl4 knockout mice, especially the female heterozygous mice. The compounds can be either food supplements or drug candidates, and can be used in the prevention and treatment of retinal diseases such as Stargardt-like dominant macular dystrophy and diseases of central nervous system.

The present invention provides an assay for determining the effect of a compound on Elov4. The assay comprises providing a male and female mouse with heterozygous Elovl4 knockout; exposing the female mouse to a compound; breeding the mice to produce offspring; and determining the genotypes of the offspring. The compounds that result in the production of homozygous Elovl4 knockout offspring are selected for further studies as candidates for food supplements and/or drugs for the prevention and treatment of the retinal diseases and CNS diseases. According to an embodiment of the present invention, both the male mouse and female mouse are exposed to the compound.

The compounds to be tested may include PPARα agonists (e.g., fibrates), HMG-Co-A-Reductase Inhibitors (statins), or other drug candidates. This group of compounds may also include dietary supplements and natural products such as gamma-linolenic acid, alpha-linolenic acid, eicosapentaenoic acid, docosahexaenoic acid, primrose oil, borage oil, blackcurrant oil. Drug candidates can be administered in such oral forms as tablets, capsules (each of which includes sustained release or timed release formulations), pills, powders, granules, elixers, tinctures, suspensions, syrups, and emulsions. Likewise, they may be administered in intravenous (bolus or infusion), intraperitoneal, subcutaneous, or intramuscular form, all using forms well known to those of ordinary skill in the pharmaceutical arts. Dietary supplements can be added into the food for the transgenic animals.

Moreover, if the compounds capable of rescuing the lethality could be selected, viable homozygous Elovl4 knockout mice could also be produced. Mouse model with homozygous deletion of the Elovl4 gene is uniquely suitable for selection of drugs and supplements for treatment and prevention of retinal disease. Homozygous deletion of the Elovl4 gene results in lethality. Further studies can determine whether this lethality is late embryonic or early postnatal. Drug candidates or dietary supplements that rescue the lethality, preserve retinal morphology and prevent the loss of vision in Elovl4 −/− animals may be effective in preservation of vision in patients with AMD and other forms of retinal disease.

The generation of Elovl4 deficient transgenic non-human animals, including mice, aids in defining the in vivo function(s) of Elovl4. As an alternative the method disclosed in section 3. 1, such Elovl4 null animals can also be used as a strain for the insertion of human ELOVL4 genes, and provides an animal model useful in the design and assessment of various approaches to modulating ELOVL4 activity and expression.

The creation of an Elovl4 null allele provides a biological platform for studies that involve future structure function analysis of Elovl4 function. Knowledge of the null phenotype provides both a basis and an impetus for production of conditional null alleles of Elovl4. The knowledge and strategy gained from the present invention provides necessary and useful information for the design and production of conditional alleles.

3.3. Screening of Triglyceride-Lowering Drug Candidates

As discussed above, dietary DHA is thought to suppress endogenous VLCFA synthesis, and thereby lower the risk of hypertriglyceridemia and cardiovascular disease. Because normal allele of Elovl4 may be involved in one of the elongation steps during DHA synthesis, inactivation of the Elovl4 gene would predictably lead to decrease in production of DHA and subsequent loss of DHA-mediated decrease in triglycerides.

As disclosed above, Elovl4 −/+ mice have increased levels of triglycerides. Drug candidates or dietary supplements that lower triglyceride levels in Elovl4 −/− and Elovl4 −/+ animals may be effective in treating hyperlipidemia in humans. Drug candidates with triglyceride lowering activity include but are not limited to niacin, pantethine, fibrates (PPARalpha agonists), statins, other drugs and their combinations.

Testing may be performed in Elovl4 −/+ or in Elovl4 −/− animals after the embryonic lethality of Elovl4 −/− is rescued and mice are switched to a normal diet. Potential triglyceride-lowering compounds can be administered in such oral forms as tablets, capsules (each of which includes sustained release or timed release formulations), pills, powders, granules, elixers, tinctures, suspensions, syrups, and emulsions. Likewise, they may be administered in intravenous (bolus or infusion), intraperitoneal, subcutaneous, or intramuscular form, all using forms well known to those of ordinary skill in the pharmaceutical arts. Effect of drug candidates is monitored by their ability to lower TG levels in plasma.

3.4. Screening of Modifiers for Elovl4 Functions

The present invention also provides a basis for performing genetic and/or pharmacological screens to modify the lethal phenotype. Such screens have been used in identifying biochemical pathways and new or novel genes that are suitable as potential drug targets. Mice with this genetic alteration within the Elovl4 gene can be used in a screen to identify new genes that modify the Elovl4 −/− or Elovl4 −/+ phenotypes. Different mouse transgenic models, such as containing copies of the human ELOVL4 gene with 797-801ΔAACTT or 796ΔT/800ΔT mutations can also be used. Such modifier genes will be expected to interact, directly or indirectly, with biosynthesis of polyunsaturated fatty acids. Alternatively, modifier genes may increase photoreceptor survival without affecting fatty acid biosynthesis. In this case new genes modifying the disease phenotype will define novel pathways important for survival of photoreceptor cells.

To begin a dominant modifier screen, N-ethyl-N-nitrosourea (ENU)-mutagenized C57BL/6J mice will be bred onto an Elovl4 −/− or Elovl4 −/+ background. Viable Elovl4 −/− mice will be identified as putative mutants carrying dominant modifying mutations conferring survival of animals. Further breeding and secondary phenotypic screens will be performed before the process of chromosome positioning and eventual gene identification. N-ethyl-N-nitrosourea (ENU) mutagenesis and subsequent genetic mapping of the modifier gene is a very well established and widely used technique (See, e.g., Vitaterna, M. H. et al Science 264:719-725 (1994)).

The following examples are presented by the way of illustration and, because various other embodiments will be apparent to those in the art, the following is not to be construed as a limitation on the scope of the invention.

### EXAMPLES

Deltagen (San Carlos, Calif.) was hired to generate the Elovl4 knockout mouse model. The company provides fee-for-services in the development and analysis of knockout mice under their DeltaSelect™ program.

### Example 1

### Construction of Elovl4 Gene Targeting Vector

From the knowledge of the genomic organization of mouse Elovl4 gene with regard to restriction sites and the exon 2, a gene targeting vector for inactivating the Elovl4 gene was prepared using standard cloning techniques (Sambrook et al., Molecular Cloning, A Laboratory Manual, Cold Spring Harbor Laboratory Press, 1989).

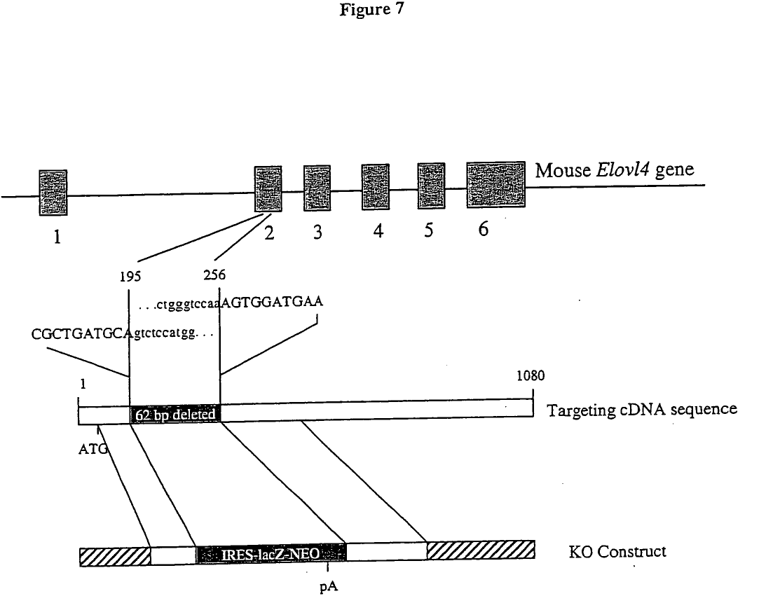

Referring to FIG. 7, the targeting vector contains from left to right: a sequence of 5′ homology with the Elovl4 locus; a IRES-lacZ-NEO expression cassette inserted in the same orientation to the Elovl4 gene to replace a fragment of 62 bp in the exon 2 of Elovl4 gene; a fragment homologous to the 3′ part of the Elovl4 gene. Targeted recombination between the vector and the wild-type Elovl4 locus results in a deletion in the Elovl4 gene including exons 2 to 6 followed by its replacement with the neomycin resistance gene.

### Example 2

### Targeted Disruption of the Elovl4 Gene in Murine ES Cells

The targeting vector of Example 1 was used in Elovl4 gene disruption experiments. The vector was linearized and electroporated into ES cells derived from 129/OlaHsd mouse substrain, and drug-resistant clones were selected. ES cell DNA from the wild-type ES cells and the clones were digested with restriction enzyme BamHI, EcoRI, and EcoRV, which cut outside of the construct arms (Construct arms are the sequences used for homologous recombination, they flank the sequence that has to be deleted; In FIG. 7, construct arms in the targeting vector are depicted by open bars flanking IRES LacZ-neo). The digested DNA was then analyzed by Southern hybridization, probing with a radio-labeled DNA fragment that hybridizes outside of and adjacent to the construct arms. The parent ES lines showed single band representing the endogenous (wild-type) allele. In contrast, the ES line that showed a second band representing the targeted allele from expected homologous recombinant event. The results were used to identify the ES cell clones carrying the targeted allele.

The targeted embryonic stem cell clones were identified using the Southern hybridization, and used for further experiments.

### Example 3

### Generation of Heterozygous Elovl4 Knockout Mice

Chimeric mice were generated with the injection of the targeted clones into the blastocysts of the 129/OlaHsd mouse substrain, using routine techniques. The chimeric mice were bred to wild-type C57BL/6 female mice. To determine the Elovl4 genotypes, genomic DNA was purified from about 1 cm of tail taken from each mouse. Southern hybridization analysis, described above, was used to confirm offspring which contained the disrupted Elovl4 allele. ES cell DNA from the wild-type ES cells and the clones were used as controls.

From these transgenic offspring, heterozygous for the Elovl4 disruption were identified, including four heterozygous mutant females and three heterozygous mutant males. Each of the identified mutants contained one copy of the altered murine Elovl4 allele (heterozygous mice, or Elovl4 +/−).

### Example 4

### Homozygous Elovl4 Knockout Mice Were Not Identified

The heterozygous Elovl4 knockout mice of Example 4 were mated with each other to generate mice in which both copies of the Elovl4 gene encoded the targeted, altered Elovl4 allele (homozygous mice, or Elovl4 −/−). It was predicted that one fourth of the mice would be homozygous for the altered Elovl4 gene, provided such mice were viable.

In order to establish homozygosity, PCR-based genotyping were performed using the following oligonucleotide primers:

(SEQ ID NO:9)

5′- CTC CGC AGA TAA ACG TGT AGC AGA C - 3′

(SEQ ID NO:10)

5′- AGA GTG CCG TTA ACA AAC CTA CCT C - 3′

(SEQ ID NO:11)

5′- GGG TGG GAT TAG ATA AAT GCC TGC TCT - 3′

PCR-generated bands will be used for establishing homozygosity, heterozygosity, or absence of the targeted deletion (as shown below).

| Homozygous | Heterozygous | WT |
|---|---|---|
| ˜406 bp | ˜406 bp | — |
| — | ˜217 bp | ˜217 bp |

Weaned progeny from the heterozygous mating were genotyped. No homozygous mutant mice were identified by PCR, whereas wild-type and heterozygous mutant mice were present. The genotypic ratio suggests an embryonic lethal phenotype of homozygous Elovl4 knockout mutant.

### Example 5

### Heterozygous Elovl4 Knockout Mice Have Higher Average Triglyceride Level Than Wild Type

Serum samples from four wild-type control animals and seven heterozygous mutant mice of approximately seven weeks of age were evaluated by a clinical biochemistry panel. Heterozygous mutant Elovl4 −/+ mice may express half the amount of Elovl4 protein as the wild-type mice. Average triglyceride level in the group of wild-type mice was 69.8 mg/dL while the average triglyceride level in the group of Elovl4 −/+ animals was 124 mg/dL (1.8 fold increase in Elovl4 −/+ animals).

### Example 6

### Selection of Dietary Supplement Formulations for the Treatment and Prevention of Retinal Diseases

The animal model of the present invention can be used in selecting a dietary formulation that can be used for treatment and/or prevention of AMD, and other retinal diseases.

Although only DHA seems to be important for retinal function, both DHA and ARA may be important for survival of homozygous Elovl4 −/− mice. In dietary supplementation studies both types of fatty acids will be supplemented to animals. Dietary supplements containing DHA and ARA are DHASCO® and ARASCO® oils, respectively, produced by Martek Biosciences Corporation (Columbia, Md.). DHASCO® and ARASCO® oils are approved for use in humans and extensively tested in rodents (Bums, R. A., et al. Food and Chemical Toxicology 37:23-36 (1999); Arterbum, L. M. et al. Food and Chemical Toxicology 38:35-49 (2000)). After 2:1 blend of ARASCO®/DHASCO® is made it will be mixed with the certified rodent diet No. 5002 (PMI Feeds Inc.) to give 120 g/kg concentration of ARASCO®/DHASCO®. The whole ARASCO/DHASCO/No. 5002 chow will be custom made and ordered from Research Diets Inc. (New Brunswick, N.J.)

Two to three Elovl4−/+ males and 2-3 Elovl4−/+ females will be given ARASCO®/DHASCO®/No. 5002 diet 1 week before male-female mating pairs are assembled in 2-3 mating cages. ARASCO®/DHASCO®/No. 5002 diet will continue to be given after male-female pairs are transferred to mating cages and while animals are maintained in mating cages. This will ensure high dietary consumption of ARA and DHA during mouse embryo preimplantation, post-implantation, and prenatal development. Females and pups will be kept on ARASCO®/DHASCO®/No. 5002 diet during the postnatal period; their litters will be given the ARASCO®/DHASCO®/No. 5002 supplementation post-weaning. Three-to four litters will be generated from each mating pair over the period of 3-4 months giving a total of 8-12 litters being fed on an ARASCO®/DHASCO®/No. 5002 diet. Similar number of mating pairs (2-3) will be assembled in control cages and will be a given standard diet.

Success of the dietary supplementation will be judged by the ability of Elovl4−/+ breeding pairs to produce viable Elovl4−/− offspring. In order to establish homozygosity, PCR-based genotyping will be performed to determine homozygosity, heterozygosity, or absence of the targeted deletion, as shown above.

After homozygosity is established by PCR-based genotyping, morphology of the retina in Elovl4−/− animals will be examined histologically at different stages of postnatal development and compared with the morphology of age-matched wild type controls to determine the beneficial effect of dietary supplementation on retinal structure.

Other embodiments are within the following claims. While several embodiments have been shown and described, various modifications may be made without departing from the spirit and scope of the present invention. Publications cited herein and the material for which they are cited are specifically incorporated by reference.

1. A transgenic mouse whose genome is heterozygous for a disruption of a native Elovl4 gene, wherein the transgenic mouse produces an average triglyceride level at about 1.8 fold of that of a wild type mouse homozygous for the native Elovl4 gene.

2. A cell line established from a transgenic mouse of claim 1.

3. The mouse of claim 1 wherein the transgenic mouse is fertile.

4. A method for producing a mouse whose genome is heterozygous for a disruption of a native Elovl4 gene, the method comprising:
a) providing a DNA sequence which targets and inserts a disruption into a Elovl4 gene;
b) introducing the DNA sequence into mouse ES cells;
c) selecting those mouse ES cells whose genome comprise a disruption of a Elovl4 gene;
d) introducing an ES cell selected in step c) in a mouse blastocyst;
e) transplanting the blastocyst of step d) into a foster mother mouse;
f) developing the transferred blastocyst to term to produce chimeric mice; and
g) mating the chimeric mice to produce a mouse heterozygous for a disruption of the Elovl4 gene;
wherein the heterozygous mouse produces an average triglyceride level at about 1.8 fold of that of a wild type mouse homozygous for a native Elovl4 gene.

5. A transgenic mouse of claim 1 wherein the genome further comprises a transgene comprising a DNA sequence encoding a non-native Elovl4 operably linked to a promoter selected from the group consisting of neural and neuronal specific promoters, wherein the mouse is viable.

6. The transgenic mouse of claim 5 wherein the promoter is selected from the group consisting of mouse Elovl4 promoter, mouse rod opsin promoter, and mouse IRBP promoter.

7. The mouse of claim 5 wherein the non-native Elovl4 is a human ELOVL4.

8. The mouse of claim 5 wherein the non-native Elovl4 is a mutant of human ELOVL4.

9. The mouse of claim 8 wherein the mutant encodes SEQ ID NO:4.

10. The mouse of claim 8 wherein the mutant encodes SEQ ID NO:6.

11. An assay for determining the effect of a compound on Elov4 comprising:
providing a male and female mouse of claim 1;
exposing the female mouse to a compound;
breeding the male and female mouse to produce offspring; and
determining the genotypes of the offspring.

12. The assay of claim 11 wherein the exposing is feeding.

13. The assay of claim 11 wherein the exposing is administering.

14. The assay of claim 13 wherein the compound is administered intramuscularly, intravenously, subcutaneously, orally, rectally, or percutaneously.

15. The assay of claim 11 wherein the compound is DHA or ARA.

16. An assay for screening a compound capable of lowering triglyceride level comprising:
exposing the mouse of claim 1 to a compound; and
determining the triglyceride level of the mouse.

17. The assay of claim 16 wherein the compound is selected from the group consisting of niacin, pantethine, fibrates (PPARalpha agonists), and statins.
